(19) United States
(12) Patent Application Publication
(10) Pub. No.: US 2022/0339601 A1
(43) Pub. Date: Oct. 27, 2022

(54) METAL-ORGANIC FRAMEWORKS FOR p-Cresyl SULFATE ADSORPTION

(71) Applicant: Colorado State University Research Foundation, Fort Collins, CO (US)

(72) Inventors: Melissa Reynolds, Fort Collins, CO (US); Jamie Cuchiaro, Fort Collins, CO (US); Nickolas Schaffner, Oskalooska, IA (US); Jon Thai, Fort Collins, CO (US)

(21) Appl. No.: 17/729,940

(22) Filed: Apr. 26, 2022

Related U.S. Application Data
(60) Provisional application No. 63/179,599, filed on Apr. 26, 2021.

(51) Int. Cl. B01J 20/22; G01N 15/08; B01D 15/00

(52) U.S. Cl. CPC B01J 20/226; G01N 15/0893; B01D 15/00; B01J 2220/46; B01J 2531/842; G01N 2015/0866

(57) ABSTRACT

Provided herein is a method for removing uremic toxins from blood is provided. The method includes exposing blood to iron-based metal-organic frameworks; and allowing the metal-organic frameworks to bind a least one uremic toxin in the blood.

### CROSS-REFERENCE TO RELATED APPLICATION

This application claims priority to U.S. Provisional Application No. 63/179,599, filed Apr. 26, 2021, which is herein incorporated by reference in its entirety.

### GOVERNMENT LICENSE RIGHTS

This invention was made with government support under grant R01 HL140301 awarded by the National Institutes of Health. The government has certain rights in the invention.

### TECHNICAL FIELD

The current invention relates to method of removal of toxins from biological fluids using metal-organic frameworks (MOFs). In some embodiments, MOFs are used to remove uremic toxin p-cresyl sulfate for example during a dialysis treatment.

### BACKGROUND

Advanced-stage chronic kidney disease necessitates routine dialysis treatment to filter patients' blood when less than 20% of kidney functionality remains. Uremia occurs when p-cresyl sulfate, indoxyl sulfate, and other uremic toxins are retained in the blood due to poor kidney function. Current dialysis technologies utilize semipermeable, artificial membranes to remove uremic toxins via reverse-osmosis. Development of new dialytic materials to remove uremic toxins is critical to improvement of point-of-care treatment.

### SUMMARY

In Example 1, a method for removing uremic toxins from blood is provided. The method includes exposing blood to iron-based metal-organic frameworks; and allowing the metal-organic frameworks to bind a least one uremic toxin in the blood.

Example 2 is the method of Example 1, wherein after allowing the metal-organic frameworks to bind the least one uremic toxin, at least 70 wt % of p-cresyl sulfate is present in a bound state.

Example 3 is the method of Example 1 or Example 2, wherein the metal-organic frameworks are iron-based metal-organic frameworks.

Example 4 is the method of any one of Examples 1-3, wherein the metal-organic frameworks include Iron 1,3,5-benzenetricarboxylate (MIL-100(Fe)).

Example 5 is the method of any one of Examples 1-4, wherein the metal-organic frameworks are Iron 1,3,5-benzenetricarboxylate (MIL-100(Fe)).

Example 6 is the method of any one of Examples 1-5, wherein the metal-organic frameworks have benzenetricarboxylate (BTC) linkers.

Example 7 is the method of any one of Examples 1-6 wherein the blood is exposed to about 700 milligrams (mg) to about 800 mg of the metal-organic frameworks.

Example 8 is the method of any one of Examples 1-7, wherein after the metal-organic frameworks bind the at least one uremic toxin, the metal-organic frameworks are separated from the blood.

Example 9 is the method of any one Examples 1-8, wherein the uremic toxin includes p-cresyl sulfate.

Example 10 is the method of Example 9, wherein the method is performed until the p-cresyl sulfate concentration of the blood is equal to or less than 10 μM.

Example 11 is a method for predicting the adsorptive capacity of a metal-organic framework for a uremic toxin, the method including determining uptake of the uremic toxin by the metal-organic framework as a function of mass of metal-organic frameworks while holding concentration of the uremic toxin in a solution and volume of the solution constant; determining uptake of the uremic toxin by the metal-organic framework as a function of concentration of the uremic toxin in the solution while holding the mass of metal-organic frameworks and volume of the solution constant; determining uptake of the uremic toxin by the metal-organic framework as a function of the solution volume while holding the mass of metal-organic frameworks and the concentration of the uremic toxin of the solution constant; and performing multivariate linear regression to produce an uptake function as an effect of the solution volume, uremic toxin content and metal-organic framework mass.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a diagram of a human patient experiencing a dialysis treatment.

FIG. 2 illustrates the structure of the metal node, organic linker, and framework of (A) MIL-100(Fe), (B) MOF-808, and (C) NU-1000 (hydrogens are omitted for clarity.)

FIG. 3 shows the comparative uptake between MIL-100(Fe)′ and MOF-808′ and those previously reported.

FIGS. 4A-4D plot p-Cresyl sulfate uptake per milligram of MOF against BET surface area, number of aromatic carbons within the linker, pore diameter and largest pore window, respectively.

FIG. 5 illustrates the structure of organic linker molecules, arranged by increasing order of p-cresyl sulfate uptake by the corresponding MOFs.

FIG. 6A measures the p-cresyl sulfate uptake as an effect of solution volume with 6 mg of MIL-100′ and 100 μM p-cresyl sulfate.

FIG. 6B measures the uptake as an effect of p-cresyl sulfate molar content with 6 mg of MIL-100′ and 10 mL of solution volume.

FIG. 6C measures the uptake as an effect of mass MIL-100(Fe)′ with 10 mL of solution volume and 100 μM p-cresyl sulfate.

### DETAILED DESCRIPTION

For convenience, before further description of the present invention, certain terms used in the specification and examples are described here. These definitions should be read in light of the remainder of the disclosure and understood as by a person of skill in the art. Also, the terms “including” (and variants thereof), “such as”, “e.g.”, “i.e.” as used herein are non-limiting and are for illustrative purposes only.

The articles “a” and “an” are used herein to refer to one or to more than one (i.e. to at least one) of the grammatical object of the article. By way of example, “an element” means one element or more than one element.

“Metal organic framework” (MOF) consists of linkers coordinated to metals resulting in a 1-dimensional, 2-dimensional, or 3-dimensional structure with well-defined and repeated structural characteristics throughout the material.

“Linker” is a multidentate ligand that binds to metals through coordinate covalent bonds such that it acts as a connector between geometric centers.

“Porosity” describes the size of the void spaces in a material. The higher the void space compared to material space, the higher the porosity. Porosity can range from 0-100%.

Disclosed herein is a method for removing uremic toxins, such as p-cresyl sulfate, from a bodily fluid by exposing the bodily fluid to metal-organic frameworks (MOFs). Uremic toxins are organic or inorganic substances that accumulate in the body fluids of a patient with, for example, acute or chronic kidney disease. In one method, a bodily fluid such as blood, blood plasma or peritoneal fluid, is exposed to the MOFs. The blood and MOFs are allowed to be in contact for a period sufficient to bind at least one uremic toxin the in blood, and then the MOFs are separated from the blood. For example, blood may be circulated through the MOFs for a period sufficient to bind the uremic toxins in the blood. In some embodiments after the sufficient period of time, at least 60 wt %, 70 wt %, 80 wt % or 90 wt % of the p-cresyl sulfate in the system is present in a bound state. Thus, when the blood and MOFs are separated, the p-cresyl sulfate content of the blood is reduced by by 60 wt %, 70 wt %, 80 wt % or 90 wt %. In some embodiments, the MOFs are in contact with the blood for at least ten seconds and less than 30 minutes. In some embodiments, the blood is circulated through the MOFs a plurality of times and during each cycle, blood is exposed to the MOFs for 10-30 seconds. The MOFs are provided in a sufficient amount.

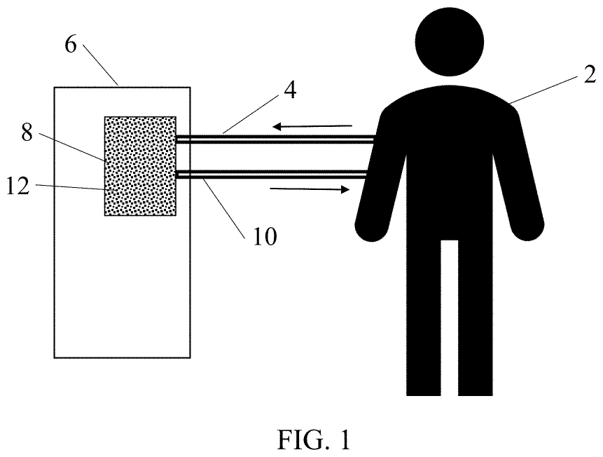

In one embodiment, the MOFs are used to absorb or remove toxins during a dialysis treatment. FIG. 1 is a diagram of a human subject or patient 2 during a dialysis treatment. During a dialysis, blood is removed from the patient 2 typically through a tube 4 connected to the patient's arm and sent to an external machine 6 that filters the blood to remove toxins, such as p-cresyl sulfate. After filtering, the blood is returned to the patient typically through a tube 10 and into the patient's arm. In some embodiments, the external machine 6 includes a container or compartment 8 known as a dialyzer. The dialyzer 8 contains the MOFs 12. Blood is removed from the patient 2, for example, from the patient's arm, and directed to the compartment 8 where it is in contact with the sufficient amount of MOFs 12 for a sufficient time to adsorb the uremic toxins. The blood is then returned to the patient through a second tube 10. A dialysis treatment is a continual flow process and blood continually flows through the process (i.e., through tube 4, dialyzer 8 and tube 10) to be returned to the patient 2. The dialysis treatment is continued for a period of time sufficient to reduce the toxins to a sufficient level. In some embodiments, the dialysis treatment is continued until the p-cresyl sulfate concentration of the blood is equal to or less than 10 μM. In some embodiments, the dialysis treatment is continued until the p-cresyl sulfate concentration of the blood is from about 5 μM. to about 10 μM.

The more efficient or quicker the toxins can be removed from the blood, the shorter the dialysis treatment or the less cycles of the dialysis treatment are required. Thus, it is desirable use MOFs that quickly remove toxins, such as p-cresyl sulfate, from the blood.

In some embodiments, the MOFs 12 are added to a commercially available dialysis machine 6. For example, the dialyzer 8 of a commercially available dialysis machine 6 can be filled with the MOFs 12. In some embodiments, as little as about 700 mg to about 800 mg MOF is needed per treatment. In one example 726 mg of FeBTC, a MOF which is composed of iron nodes and a 1,3,5-benzenetricarboxylate (BTC) linker, may be needed per dialysis treatment for an adult patient having an pre-treatmentp-cresy/sulfate concentration of 120 μM, assuming the average adult has an average of 2.75 L of plasma.

In some embodiments, an iron-based MOF is used for the adsorption of uremic toxins, such as p-cresyl sulfate, by exposing the iron-MOF to the bodily fluid. Metal-organic frameworks (MOFs) have high porosity and surface area. Suitable MOFs are not fully saturated. That is, the MOFs contain open sites. These open sites bind the uremic toxins during use.

MOFs are composed of organic linker molecules coordinated to metal nodes in a repeating array. MOFs offer unparalleled tunability in comparison to other solid-state materials such as zeolites due to the broad array of metal and organic substituent units. As of 2019, there were more than 82,600 known MOFs in the Cambridge Structural Database compared to just 248 zeolites in the Database of Zeolite Structures. MOFs are highly porous and feature high surface areas per volume in comparison to other solids, making them excellent materials for adsorption. Their high adsorptive capacity is well documented for gas storage and heavy metal capture.

In some examples, suitable MOFs have large pore apertures. In some embodiments, suitable MOFs are mesoporous material which have pores about 2-50 nm in diameter. Suitable MOFs can additionally or alternatively have large linkers, such as 1,3,5-benzenetricarboxylate (BTC). Illustrative MOFs include Iron 1,3,5-benzenetricarboxylate (Fe(BTC)).

Suitable MOFs are bio-stable. That is, the MOF is stable or does not decompose in the bodily fluid. Suitable MOFs also non-reversibly bind the uremic toxins, and preferably selectively and non-reversibly bind such toxins so that the MOFs do not adversely affect the fluid.

One example iron-based MOF is Iron 1,3,5-benzenetricarboxylate (MIL-100(Fe)), which is composed of iron nodes and a 1,3,5-benzenetricarboxylate (BTC) linker. MIL-100(Fe) is crystalline and has a reported Brunauer, Emmett and Teller (BET) surface area of 1024-2200 m² g⁻¹ and internal cage diameters of 25 and 29 Å. The BET surface area is dependent on particle size and includes both the internal and exterior surface area, and much of the MOFs' surface area is found on interior particle surfaces. MIL-100(Fe) is stable in liquid water. MIL-100(Fe) is less toxic than zirconium-based MOFS because of to its iron—(rather than zirconium—) based metal nodes. Krict F100 is a commercially available iron-based MOF and modified version of MIL-100(Fe) available from Korea Research Institute of Chemical Technology (KRICT), Daejeon, South Korea. Krict F100 is available in a two forms, dark brown hydrated form and a pale brown dehydrated form.

In some embodiments, the MOF can be a zirconium-based MOF such as zirconium 1,3,5-benzenetricarboxylate (MOF-808). MOF-808 is composed of zirconium nodes and a 1,3,5-benzenetricarboxylate (BTC) linker.

The method can use a single type of MOF, such as MIL-100(Fe) or a mixture of MOFs which may have the same or different metals. For example, the mixture can include a mixture of iron-based MOFs (a mixture of same metal-type MOFs) or a mixture of iron-based and zirconium-based MOFs (a mixture of different metal-type MOFs).

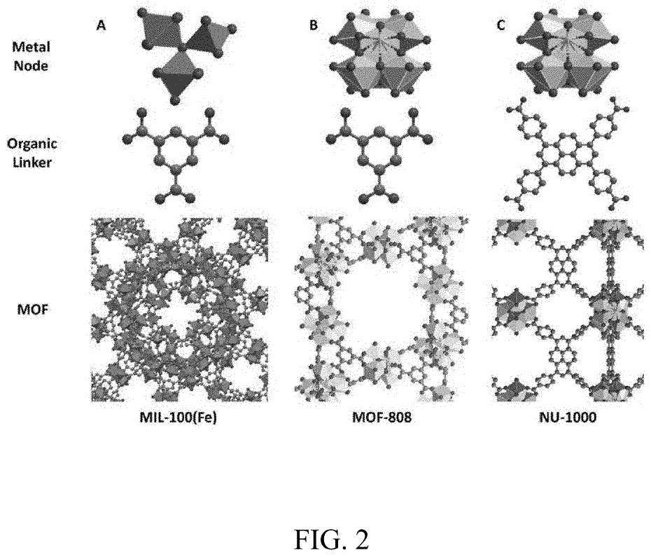

The parametric effects of MOFs on adsorptive uptake from solution was determined. This information can be used to select suitable MOFs for the method described herein. More specifically, the adsorptive uptake from solution was compared for MIL-100(Fe), MOF-808, and NU-1000. MOF-808 is a zirconium trimesate analog and was used as control for comparison with the iron-based MIL-100(Fe). Although MOF-808 and MIL-100(Fe) have the same organic linker, MIL-100(Fe) and MOF-808 have different structural geometry at the metal nodes. The zirconium(IV) forms Zr₆O₈ clusters capped by hydroxide and BTC anions within MOF-808, while Fe(II/III) centers have octahedral geometry and form oxocentered trimers connected by BTC anions. Although the metal atoms have similar connectivity, they are coordinated to different functional groups and therefore would presumably behave differently. The structures of the metal node, organic linker, and framework for MIL-100(Fe), MOF-808, and NU-1000 are shown in FIG. 2. Both MIL-100(Fe) and MOF-808 have a 1,3,5-benzinetricarboxylate linker. Both MOF-808 and NU-1000 have a Zr₆ node. MIL-100(Fe) has oxocentered Fe trimers for a metal node, and NU-1000 has a tetratopic pyrene-based linker.

The high porosity of the MOFs causes them to have BET high surface areas, and compositional differences between metal and organic sites allows for various adsorptive pathways. BET can be measured by degassing a MOF sample to eliminate guest molecules in the framework. Then an inert gas (such as nitrogen) is adsorbed to the MOF. The pressure in the sample tube is measured as gas is added, and the difference between actual and theoretical pressure is attributed to gas adsorbed to the sample.

Also described herein is a method for predicting the adsorption capacity of a MOF. Emphasis on rationalizing differences in adsorption has previously been placed on BET and internal MOF cage diameter. Prior to work described herein it was believed that p-cresyl sulfate adsorption linearly correlated to one or both parameters. The high surface area characteristic of MOFs is due to their high porosity. In addition to hydrophobic interactions between the organic linker and nonpolar regions within the adsorbate, there is the possibility of coordination of adsorbate with vacant metal sites, or diffusion and entrapment of adsorbate within the MOF. Given that MOF-808 and MIL-100(Fe) have the same organic linker, one would have expected that adsorption between MIL-100(Fe) and MOF-808 would be very similar. However, as described herein, the adsorption for these MOFs is not similar.

In theory, the large surface area will facilitate adsorptive separation of target molecules. The difficulty is in designing a MOF with specificity for target molecules, and that most of the surface area is unavailable for adsorption if the adsorbates can't pass through the pore apertures.

A method for predicting the adsorptive capacity of a metal-organic framework for a uremic toxic includes determining uptake of the uremic toxin by the MOF as a function of mass of the MOF, determining uptake of the uremic toxin by the MOF as a function of concentration of the uremic toxin, determining uptake of the uremic toxin by the MOF as a function of the solution volume, and performing multivariate linear regression to produce an uptake function as an effect of the solution volume, uremic toxin content and MOF mass. In some embodiments, the uremic toxin can be p-cresyl sulfate. In some embodiments the MOF can be a iron-based MOF such as FE(BTC).

The uptake of the uremic toxin by the MOF is determined as a function of mass of MOF. For example, this measurement can be determined by creating a solution of the uremic toxin, such as p-cresyl sulfate, varying amount or mass of MOF added to the solution and measuring the uptake of the uremic toxin by the MOF. In this example, the concentration of the uremic toxin and volume of the solution are held constant. The results can be plotted as a linear plot where the slope of the trendline indicates the uptake as a function of the mass of MOF.

The uptake of the uremic toxin by the MOF is also determined as a function of concentration of the uremic toxin. For example, this measurement can be determined by creating solutions having varying concentrations of the uremic toxic, adding the same amount or mass of MOF to the solutions and measuring the uptake of the uremic toxin by the MOF. In this example, the mass of MOF added to solution and volume of solution are held constant. The results can be plotted as a linear plot where the slope of the trendline indicates the uptake as a function of the concentration of the uremic toxin.

The uptake of the uremic toxin by the MOF is also determined as a function of the solution volume. For example, this measurement can be determined by creating solutions the uremic toxin of varying volumes, adding the same amount or mass of MOF to the solutions and measuring the uptake of the uremic toxin by the MOF. In this example, the mass of MOF and the concentration of the uremic toxin of the solution are held constant. The results can be plotted as a linear plot where the slope of the trendline indicates the uptake as a function of the volume of the uremic toxin solution.

After the above determinations are complete, a multivariate linear regression is performed to product an uptake function as an effect of the solution volume, uremic toxin content and the MOF mass.

### Comparison of Synthesized MIL-100(Fe), MOF-808, and Zr-MOFs' p-Cresyl Sulfate Uptake

MIL-100(Fe) and MOF-808 were synthesized according to known methods. The MOFs (which are designated MIL-100(Fe)' and MOF-808′) were then subjected to the adsorption method using p-cresyl sulfate in water as described in Kato, S.; Otake, K.; Chen, H.; Akpinar, I.; Bum, C. T.; Islamoglu, T.; Snurr, R. Q.; Farha, O. K. Zirconium-Based Metal—Organic Frameworks for the Removal of Protein—Bound Uremic Toxin from Human Serum Albumin. J. Am. Chem. Soc. 2019, 141 (6), 2568-2576, which is herein incorporated by reference in its entirety. The temperature, exposure time, and solution concentration used were identical to that of Kato. The MOF mass and solution volume were scaled up by a factor of 4 as compared to Kato.

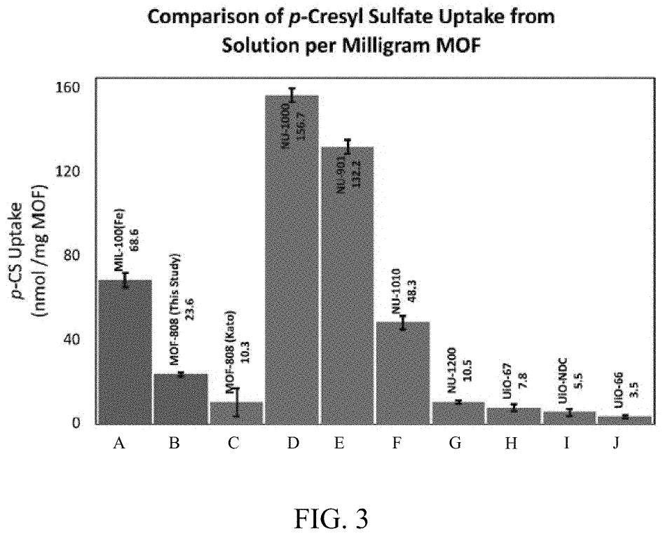

The chromatographic p-cresyl sulfate peak area of a 100 μM stock solution was compared to analytical samples treated with MIL-100(Fe)′ and MOF-808′. FIG. 3 shows the comparative uptake between MIL-100(Fe)′ and MOF-808′ and those reported by Kato et al. Variation is reported as the standard deviation of samples, n =4 (MOF-808) and n =8 (MIL-100). In FIG. 3, A represents uptake of MIL-100(Fe)′ of the present study, B represents uptake by MOF-808′ of the present study, and C-J are those reported by Kato et al. (C is MOF-808, D is NU-1000, E is NU-901, F is NU-101, G is NU-1200, H is UiO-67, I is UiO-NDC and J is UiO-66).

The equilibrium concentration of p-cresyl sulfate decreased in the presence of both MOF-808′ and MIL-100(Fe)', indicating adsorption to the MOF. MOF-808′ removed 13 nmol/mgp-cresyl sulfate more than was previously reported for the same material. That is, MOF-808′ took up p-cresyl sulfate approximately twice as efficiently than previously reported, per milligram of MOF. MIL-100(Fe)′ took up approximately three times more p-cresyl sulfate than MOF-808′, per milligram of MOF. The apparent difference in uptake between MOF-808′ data and literature value may be the result of structural differences between syntheses such as the number of formate ions capping the nodes, effects from scaling up sample prep by a factor of 4, or inconsistencies in particle size between the two studies. The average particle diameter for the MOFs synthesized was 74 ±12 nm for MOF-808′ and 213 ±146 nm for MIL-100(Fe)′.

Between the MOFs synthesized herein, p-cresyl sulfate uptake was approximately three times greater for MIL-100(Fe)′ than MOF-808′ on a per milligram MOF basis. In fact, MIL-100(Fe)′ adsorbed more p-cresyl sulfate than 75% of MOFs per milligram of MOFs previously reported. This result was surprising, as it was expected that smaller particle size samples (MOF-808′) would adsorb more p-cresyl sulfate.

Table 1 shows the p-cresyl sulfate uptake, BET surface area, number of aromatic carbons in the linker, largest cage diameter, and largest pore window aperture of MIL-100(Fe)′ and 8 Zr-based MOFs (including MOF-808′).

TABLE 1
Comparison of MOFs^{a}

| | | | | Largest | Largest |
|---|---|---|---|---|---|
| | p-CS uptake | | Aromatic | cage | pore |
| | (nmol mg⁻¹ | BET surface | carbons in | diameter | window |
| MOF | MOF)¹³ | area (m² g⁻¹)¹³ | linker^{b} | (Â)¹³ | (Â) |
| NU-1000 | 156.7 | 2140 | 40 | 29.5 | 10²⁶ |
| NU-901 | 131.7 | 2345 | 40 | 12 | 12²⁷ |
| MIL- | 68.6^{b} | 2200¹⁸ | 6 | 29¹⁸ | 8.6²⁴ |
| 100(Fe)^{b} | | | | | |
| NU-1010 | 48.3 | 1780 | 36 | 29.5 | |
| MOF-808^{b} | 23.6^{b} | 1710 | 6 | 17.2 | 10²⁸ |
| NU-1200 | 10.5 | 2105 | 28 | 21.6 | |
| MOF-808¹³ | 10.3 | 1710 | 6 | 17.2 | 10²⁸ |
| UiO-67 | 7.8 | 2505 | 12 | 23.4 | 8²⁹ |
| UiO-NDC | 5.5 | 1960 | 10 | 20 | 7³⁰ |
| UiO-66 | 3.5 | 1685 | 6 | 15.9 | 7³¹ |

^{a}Comparison includes BET surface area, number of aromatic carbons in the MOF linker, largest cage diameter, and largest pore window in order of decreasing p-cresyl sulfate uptake. Our MIL-100(Fe) and MOF-808 were characterized by PXRD, and BET values may be different than those cited. ^{b}Present study.

p-Cresyl sulfate uptake per milligram of MOF was plotted against each parameter in the table in FIGS. 3A-3D. The coefficients of determination (R2) for FIGS. 3A-3D are 0.1430, 0.7034, 0.0383, and 0.4780, respectively. Error margins are included where reported.

There is poor correlation between the BET surface area and p-cresyl sulfate uptake (FIG. 3A), suggesting that BET is a poor predictor of p-cresyl sulfate uptake. Hence, a difference in BET alone cannot account for the change in p-cresyl sulfate adsorption between MIL-100(Fe)′ and MOF-808′.

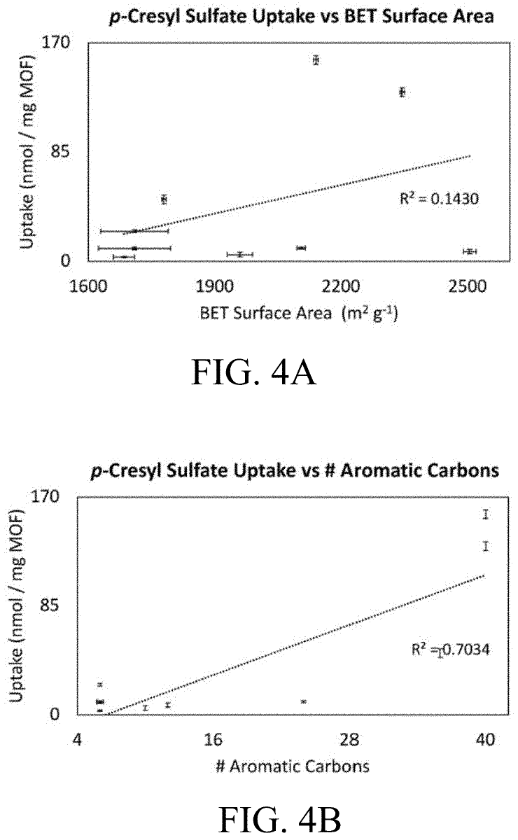

There is also poor correlation between the number of aromatic carbons within the linker and p-cresyl sulfate uptake as shown in FIG. 4B, in which the number of aromatic carbons within the linker was used as an approximation of the linker surface area. An improvement in the coefficient of determination is seen when comparing FIG. 4B and FIG. 4A (R2 =0.7034 and 0.1430, respectively). At least when comparing MOFs with the same metal nodes, the number of aromatic carbons in the linker is a better predictor of p-cresyl sulfate uptake than the BET surface area. However, the number of aromatic carbons cannot explain the difference between MOF-808′ and MIL-100(Fe)′ since they have the same organic linker.

FIG. 4C plots the p-cresyl sulfate uptake against the largest cage diameter. It was found that the cage diameter was less correlated to uptake than BET surface area (R²=0.0383). This poor correlation suggests that the internal cage diameter of a MOF is also a poor indicator of its p-cresyl sulfate adsorption capacity, even when the metal node remains constant. While not wishing to be bound, one possible theory is that while MOFs with large cage diameters have ample internal surface area to adsorb molecular species, the interior surface may be unavailable to any adsorbates unable to pass through pore windows into the interior of the framework. Thus, while theoretically active sites exist within the interior of the framework, such sites are potentially inaccessible to the substrate.

The p-cresyl sulfate molecule was digitally modeled and the distance between atoms was computed. It was found that the molecular dimensions are 8.6x4.3x2.5 Å. The longest dimension of p-cresyl sulfate is less than or equal to the pore window aperture of both MIL-100(Fe)′ and MOF-808′. It is feasible that some diffusion of p-cresyl sulfate into the MOFs could occur. As adsorption progresses, species adsorbed to sites on or closest to the exterior surface could also sterically restrict the pore aperture and hinder diffusion into the MOF interior. In this aqueous system, diffusion of p-cresyl sulfate is also limited by the diffusion of solvent through the MOF. One p-cresyl sulfate molecule is highly unlikely to diffuse through several windows and cages to the center of MOF particles in a random walk, so adsorption likely occurs primarily on external surfaces or inside pores near the surface.

FIG. 4D plots p-cresyl sulfate uptake against the largest pore window. There is poor correlation between variables but again an improvement was seen in the coefficient of determination in 3D over 3C (R²=0.4780 and 0.0383, respectively). It was expected that MOFs with pore window diameters smaller than the toxin to have no diffusion of p-cresyl sulfate into the framework, and only be able to adsorb toxin at sites on the exterior framework surface. For the MIL-100(Fe)′ and MOF-808′ materials, the largest pore window of MIL-100(Fe)′ is about 1.4 A smaller than MOF-808′, but the p-cresyl sulfate uptake of MIL-100(Fe)′ is about 3 times larger than MOF-808′. On the basis of particle size alone, there should be more pores available for adsorption in MOF-808′ than MIL-100(Fe)′. If the adsorption was driven only by cage diameter, MOF-808′ should have taken up more p-cresyl sulfate than MIL-100(Fe)′ on a per milligram basis.

The poor agreement between p-cresyl sulfate uptake with BET surface area and cage diameter indicates that adsorption occurs primarily on the exterior particle surface or in pores very near the surface of the MOFs. Adsorption occurring at the exterior particle surface would not necessarily correlate linearly with BET surface area because BET detects both exterior and unproductive interior surface area. If adsorption is limited to the exterior surface of the MOF and adsorption capacity primarily driven by surface area, then it follows that a material with smaller particles (greater exterior surface area to volume ratio) should have a greater adsorption capacity than a comparable material with larger particles. If particle size governs p-cresyl sulfate adsorption, then the 74±12 nm MOF-808′ particles should adsorb more than the 213±146 nm MIL-100(Fe)′ particles. Instead, MIL-100(Fe)′ adsorbed more p-cresyl sulfate, thus particle size alone cannot account for the observed p-cresyl sulfate adsorptive capacities of the two MOFs.

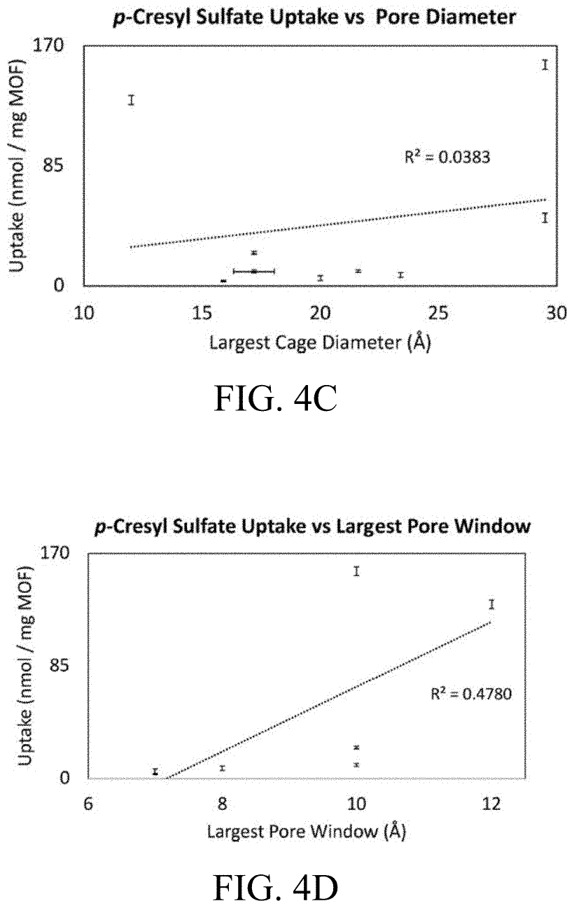

Previous work has demonstrated that the size and orientation of the aromatic systems in the linker can influence the p-cresyl sulfate uptake capacity of an MOF. FIG. 5 shows the organic linkers for MOFs in Table 1, arranged in order of increasing p-cresyl sulfate uptake (implicit hydrogens are omitted for clarity.) The aromatic regions of the linker molecules may interact hydrophobically with the aromatic region of the p-cresyl sulfate molecule during physisorption. The qualitative assumption was made that the hydrophobicity of the linker approximately increases with the number of aromatic carbons in the linker. For example: assume UiO-66 (6 aromatic carbons) is less hydrophobic than UiO-NDC (10 aromatic carbons), which is less hydrophobic than UiO-67 (12 aromatic carbons), NU-1200 (28 aromatic carbons), NU-1010 (36 aromatic carbons), and NU-901/NU-1000 (40 aromatic carbons), respectively. Thus, there is a general trend of increasing hydrophobicity of the linker with increased p-cresyl sulfate uptake, with the exception of MOF-808′ and MIL-100(Fe)′. This increasing linker aromaticity may contribute to the increased adsorptive capacity of the MOFs by increasing the surface area available for hydrophobic interactions when the composition of the metal node is held constant. However, the difference in adsorptive capacity between MOF-808′ and MIL-100(Fe)′ cannot be attributed to differences in aromaticity of the linker, indicating some additional factor is influencing adsorption capacity of the MOF. (The NU-1200 linker is unique within this group because the aromatic systems are isolated from each other in four discrete groups. The linker is composed of a central benzene which is functionalized with benzoate groups at the first, third, and fifth positions and methyl groups at the second, fourth, and sixth positions. The benzoate groups are rotated relative to the central benzene due to stearic effects of the adjacent methyl groups. The rotated conformation limits orbital overlaps between the aromatic benzoate systems and the central benzene.)

The poor agreement between the effect of BET surface area, particle size, cage diameter, pore window aperture, and linker hydrophobicity on p-cresyl sulfate uptake implies that these factors cannot account for the different uptake between MIL-100(Fe)′ and MOF-808′. While not wishing to be bound by theory, it is believed that the metal sites in MIL-100(Fe)′ can participate in adsorptive interactions with the p-cresyl sulfate more readily than zirconium centers in MOF-808′, which accounts for differences in adsorptive capacities between MIL-100(Fe)′ and MOF-808′. The formal negative charge on the terminal oxygen within p-cresyl sulfate could preferentially interact with coordinatively unsaturated metal sites on the exterior MOF surface in addition to hydrophobic interactions between p-cresyl sulfate's toluyl group and the benzyl group of the trimesate linker. Vacant coordination sites on iron atoms at the exterior surface of MIL-100(Fe)′ are capable of coordinating to sulfate groups, whereas the carboxylate-capped Zr₆O₈ clusters of zirconium-based MOFs are less available to accept an electron pair and coordinate with a sulfate group from a p-cresyl sulfate molecule. Hence, the metal-ion interactions will be much more favorable in MIL-100(Fe)′ compared to MOF-808′.

Based on the above analysis, the total adsorptive capacity of a MOF can be predicted by equation (1):

Adsₜₒₜ=(Ads_{LEX}+Ads_{NEx})+(Ads_{LIN}+Ads_{NIN}) (1)

where Adsₜₒₜ is the total adsorptive capacity of the MOF, Ads_{LEX} is the adsorptive capacity of the organic linker on the exterior surface, Ads_{NEX} is the adsorptive capacity of the metal node on the exterior surface, Ads_{LIN} is the adsorptive capacity of the organic linker on the interior surface, Ads_{NIN} is the adsorptive capacity of the metal node on the interior surface.

Investigation of p-Cresyl Sulfate Uptake as Effected by Solution Volume, p-Cresyl Sulfate Content, and Mass MIL-100(Fe)′

The contribution of (Ads_{LIN}+Ads_{NIN}) to Adsₜₒₜ is likely very small. Thus, the total adsorption may be accounted for by some combination of p-cresyl sulfate-node and p-cresyl sulfate linker adsorptive interactions.

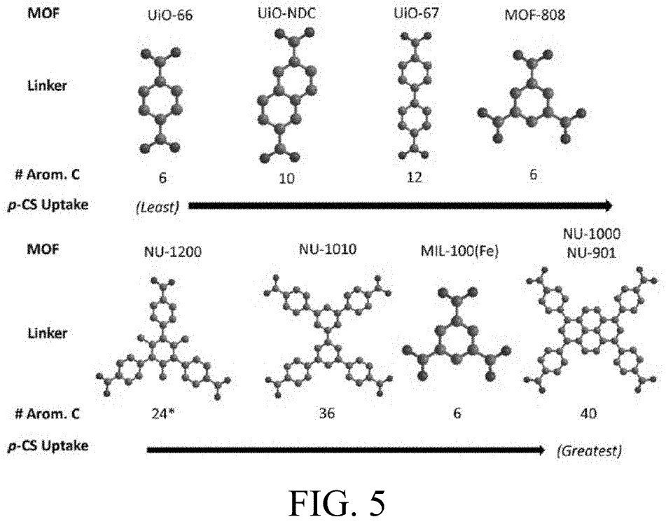

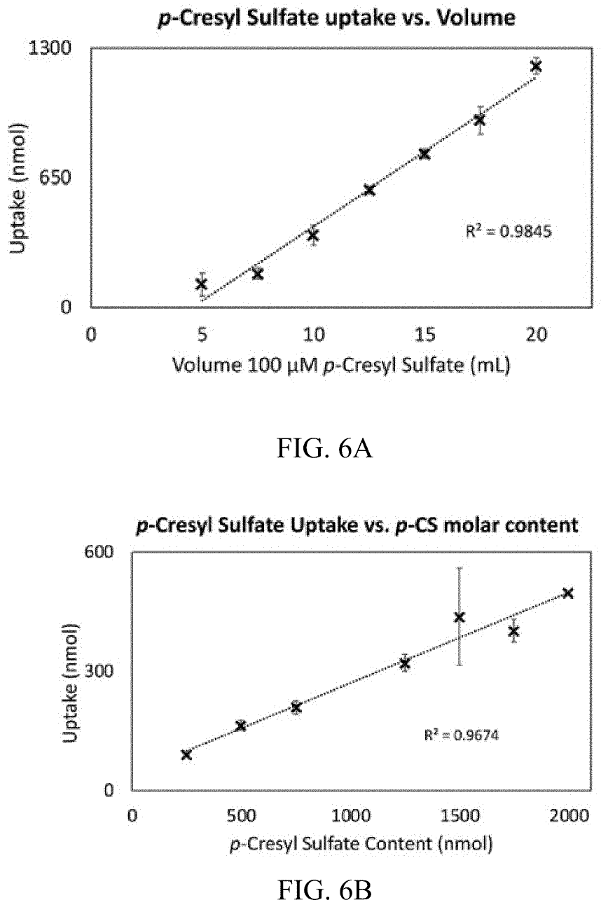

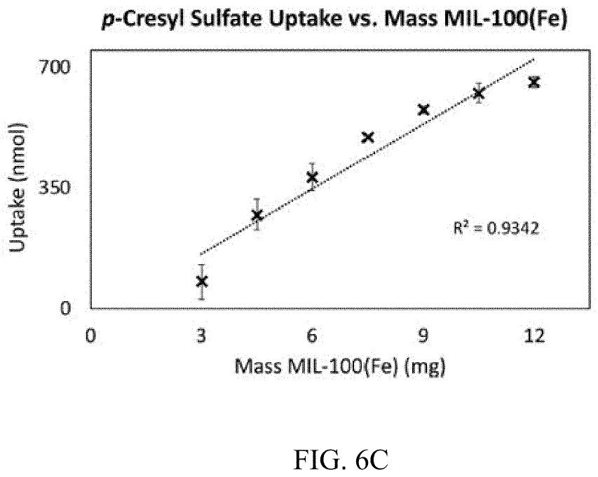

A series of experiments were performed to better understand the p-cresyl sulfate uptake by MIL-100(Fe)′. The MIL-100(Fe)′ mass, p-cresyl sulfate content, and solution volume were identified as key parameters in the sample preparation. The MIL-100(Fe)′ mass was varied while holding the p-cresyl sulfate content and volume constant and the p-cresyl sulfate uptake was measured. This method was repeated but the p-cresyl sulfate content was varied, and then again while the solution volume was varied. The p-cresyl sulfate in solution was calculated using Equation 1. In each case, the amount of p-cresyl sulfate in solution at equilibrium decreased compared to the control, indicating adsorptive uptake of p-cresyl sulfate by MIL-100(Fe)′. The result of these experiments were three linear plots where the slope of the trendline indicates the uptake as a function of the variable parameter. These plots are presented in FIGS. 6A-6C. FIG. 6A measures the p-cresyl sulfate uptake as an effect of solution volume with 6 mg of MIL-100′ and 100 μM p-cresyl sulfate; FIG. 6B measures the uptake as an effect of p-cresyl sulfate molar content with 6 mg of MIL-100′ and 10 mL of solution volume; and FIG. 6C measures the uptake as an effect of mass MIL-100(Fe)′ with 10 mL of solution volume and 100 μM p-cresyl sulfate. The three experiments were collected in triplicate measuring and data point represents the average and standard deviation of n≥3 measurements.

A multivariate linear regression analysis produced an uptake function as an effect of the solution volume, p-cresyl sulfate content, and MIL-100(Fe)′ mass. Uptake increased with solution volume in contact with the MOF and the mass of MIL-100(Fe)′, and the p-cresyl sulfate content to a smaller extent. The trendline slopes of p-cresyl sulfate uptake versus volume, p-cresyl sulfate molar content, and mass of MIL-100(Fe)′ were 57.8 nmol (mL solution)⁻¹, 0.2 nmol (nmol content)⁻¹, and 65.6 nmol (mg MOF)⁻¹, respectively. It is envisioned that similar calculations can be used to quantify the adsorptive capacity of MIL-100(Fe)′ in plasma, where inhibitive effects from albumin and other proteins is expected.

In conclusion, MIL-100(Fe) is a commercially available, water-stable MOF capable of removing uremic toxin from aqueous solution better than its zirconium analog, MOF-808. MIL-100(Fe) and MOF-808 were synthesized and their p-cresyl sulfate uptake capacity compared with zirconium MOFs previously reported for the same system. MIL-100(Fe) was observed to adsorb p-cresyl sulfate more efficiently than MOF-808 as well as 75% of previously reported Zr-based MOFs. The p-cresyl sulfate adsorption by nine MOFs was compared with respect to reported BET surface area, pore window size, cage diameter, and linker hydrophobicity approximated by the number of aromatic carbons in the linker. There is poor correlation between uptake and each of the parameters considered, but the number of aromatic carbons was the best predictor of p-cresyl sulfate uptake with an R2=0.7034 when considering MOFs with the same metal nodes. All of these parameters are poor predictors/explanations for differences in adsorptive capacity when changing the metal node identity of the MOF (MIL-100(Fe) to MOF-808). Greater observed adsorption capacity of MIL-100(Fe) compared to MOF-808 may results from direct coordination of the formally charged oxygen within the p-cresyl sulfate to metal sites in the MOF, occurring more favorably within MIL-100(Fe) than MOF-808. These results indicate a complex adsorption mechanism, where the total adsorption to the MOF is a combination of hydrophobic interaction between p-cresyl sulfate and the linker and electrostatic interaction between p-cresyl sulfate and the metal node. Solution volume, p-cresyl sulfate content, and mass of MIL-100(Fe) independently contribute to p-cresyl sulfate uptake as determined by multivariate linear regression analysis. Mass of MIL-100(Fe) was observed to have the greatest impact on p-cresyl sulfate uptake among variables considered.

### EXAMPLES

Embodiments of the present invention are further defined in the following non-limiting Examples. It should be understood that these Examples, while indicating certain embodiments of the invention, are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims. Unless otherwise noted, all parts, percentages, and ratios reported in the following examples are on a weight basis.

The following materials were used: 1,3,5-Benzenetricarboxylic acid, >98% (B004325G); N,N-dimethylformamide, >99.8% (D119-4); formic acid, >88% (A118P-500); acetonitrile, >99% (A998-4); acetone, >99.5% (A18-4); methanol, (A412-4); zirconyl chloride octahydrate, 99.9% (AA8610818); and iron(III) chloride hexahydrate, >97% (I88-500) were purchased from Fisher Scientific. Potassium p-cresyl sulfate, >98% (TCP2091-1G) was purchased from VWR. Basolite F300 (690872) was purchased from Sigma-Aldrich. Distilled water was purified by a Millipore Direct-Q water purification system to greater than 18.2 MΩ resistivity prior to use.

Synthesis of MOF-808′ was based on previously reported methods in Furukawa, H.; Cordova, K. E.; O'Keeffe, M.; Yaghi, 0. M. The Chemistry and Applications of Metal-Organic Frameworks. Science 2013, 341 (6149), 1230444-1230444, which is herein incorporated by reference in its entirety. In brief, MOF-808′ was synthesized by adding 250 mg (1.2 mmol) of 1,3,5-benzenetricarboxylic acid to 950 mg (3 mmol) of zirconyl chloride octahydrate in 40 mL of 1:1 formic acid and N,N-dimethylformamide. This mixture was sealed, sonicated for 20 minutes to dissolve, then incubated at 135 ° C. for 7 days. The resulting solid was vacuum filtered and washed three times with 10 mL of N,N-dimethylformamide (DMF), then solvent exchanged via Soxhlet exchange with acetone for 2 days. The white solid was dried at 125 ° C. under vacuum overnight and ground for 60 seconds by mortar and pestle. The powder was activated at 135 ° C. under vacuum overnight prior to use.

Synthesis of MIL-100(Fe)′ was based on previously reported methods in Sun, D. T.; Peng, L.; Reeder, W. S.; Moosavi, S. M.; Tiana, D.; Britt, D. K.; Oveisi, E.; Queen, W. L. Rapid, Selective Heavy Metal Removal from Water by a Metal—Organic Framework/Polydopamine Composite. ACS Cent. Sci. 2018, 4 (3), 349-356, which is herein incorporated by reference in its entirety. MIL-100(Fe)′ was synthesized by adding 19.4 g (72 mmol) iron(III) chloride to 6.7 g (32 mmol) 1,3,5-benzenetricarboxylic acid in 120 mL of water. This mixture was sealed, sonicated for 1 hour to homogenize, and incubated at 90 ° C. for 24 hours. The resulting solid was vacuum filtered, then washed with 100 mL of DMF followed by 100 mL of water. The solid was solvent exchanged via Soxhlet exchange with methanol for 2 days. The red-orange solid was dried at 110 ° C. under vacuum overnight and ground for 60 seconds by mortar and pestle. The powder was activated at 110 ° C. under vacuum overnight prior to use.

### MOF Characterization

Following synthesis and activation, both MOF-808′ and MIL-100(Fe)′ were characterized via powder Xray diffractometry (PXRD), Fourier-Transform Infrared spectroscopy (FTIR), and scanning electron microscopy (SEM). PXRD and FTIR spectra were verified by comparison with previously reported spectra.

A Bruker D8 Discover DaVinci Powder X-ray Diffractometer with Cu Kα radiation (λ=1.5406 Å) was used, operated at 40 kV and 40 mA. A 0.6 mm divergent slit was placed on the primary beam side and a high-resolution energy dispersive LYNXEYE-XE-T detector was placed on the diffracted beam side during the XRD studies. Samples were seated on a B-doped silicon zero-diffraction plate and irradiated with Cu Kα radiation. 2θ was set between 4 and 50° for MOF-808′ and 5-50° for MIL-100(Fe)′.

FTIR analysis was performed on a Thermo-Nicolette 6700 equipped with KBr beam splitter and diamond crystal attenuated total reflectance. Background and analytical spectra were compiled from 128 scans. Samples were analyzed from 650 to 4000 cm⁻¹.

SEM imaging was performed using a JEOL JSM-6500F microscope. An accelerating voltage of 15.0 kV and a working distance of 14.1 and 13.8 mm were used for MOF-808′ and Mil-100(Fe)′, respectively. All samples were placed under vacuum and coated with 20 nm of gold prior to imaging. Three to four representative images were taken at three different magnifications for each sample.

### p-Cresyl Sulfate Uptake

High-performance liquid chromatography (HPLC) analysis was performed on an Agilent Infinity II 1260 HPLC-DAD using an Agilent Poroshell 4.6×100 mm C18 column with 2.7 μm particle size. The mobile phase was 1:1 acetonitrile (ACN)/water with a flow rate of 0.4 mL/min under isocratic conditions. The column temperature set to 30 ° C. Sample injections were 20 μL and detection was carried out at 192 ±2 nm with a reference at 360 ±50 nm. Using this method, the p-cresyl sulfate absorbance peak was detected.

p-Cresyl sulfate stock solution was prepared fresh prior to each experiment. All samples were prepared in individual scintillation vials, including solvent, MIL-100(Fe)′, and p-cresyl sulfate blanks. MIL-100(Fe) blanks were prepared by adding 6 mg of MIL-100(Fe)′ to 10 mL of HPLC-grade water. p-Cresyl sulfate blanks were used to determine the initial absorbance peak area (mAU*min). To compare the p-cresyl sulfate uptake of our MIL-100(Fe)′ and MOF-808′ with previously published results, samples were prepared by adding 6 mg of MIL-100(Fe)′ to 10.0 mL of 100 μM p-cresyl sulfate solution. To assess the effect of MIL-100(Fe)′ mass on p-cresyl sulfate uptake, samples were prepared by adding 3-12 mg of MIL-100(Fe)′ to 10.0 mL of 100 μM p-cresyl sulfate solution. To assess the effect of p-cresyl sulfate content on uptake, samples were prepared by adding 6 mg of MIL-100(Fe)′ to 1.25-10.0 mL of 200 μM p-cresyl sulfate solution and diluted to 10 mL total volume. To assess the effect of p-cresyl sulfate solution volume on uptake, analytical samples were prepared by adding 6 mg of MIL-100(Fe)′ to 5.0-20.0 mL of 100 μM p-cresyl sulfate solution. All samples were prepared in triplicate, vortexed for 5 seconds, and incubated for 24 hours at 24 ° C. All samples were filtered after incubation through 0.45 μm PTFE syringe filters, then transferred to HPLC vials.

In a single-absorbent system, the analyte concentration is linearly related to the absorbance peak area of the analyte. It was verified that p-cresyl sulfate was the only 192 nm light absorbing species in the system via the solvent and MIL-100(Fe) blanks and assigned the theoretical stock concentration to the average peak area of the p-cresyl sulfate blank. The method linearity was confirmed via calibration curve with stock standards between 10 and 200 μM p-cresyl sulfate with an R²=0.997. The p-cresyl sulfate concentration of analytical samples was calculated using Equation 2:

$${C}_{sample} = ( 1 - \frac{{A}_{initial} - {A}_{sample}}{{A}_{initial}} ) \times {C}_{initial} \qquad ( 2 )$$

where Cₛₐₘₚₗₑ is the p-cresyl sulfate concentration (μM), Cᵢₙᵢₜᵢₐₗ is the theoretical initial concentration (μM), Aᵢₙᵢₜᵢₐₗ is the average p-cresyl sulfate blank peak area (mAU*min), and Aₛₐₘₚₗₑ is the average p-cresyl sulfate peak area of each sample replicate (n=3). Data is reported as the average ± the standard deviation.

When reporting the p-cresyl sulfate uptake data, it was delineated between the p-cresyl sulfate molar content and the p-cresyl sulfate molarity. Multivariate linear regression analysis was performed for p-cresyl sulfate uptake as an effect of MIL-100(Fe) mass, p-cresyl sulfate content, and solution contact volume. This analysis determined that all three parameters are independent of each other with respect to p-cresyl sulfate uptake.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the above described features. For example, while the embodiments described above refer to use of the polymeric composition in a medical device or for the treatment of a disease or condition, the polymeric composition may be used in other fields. For example, the polymeric composition may be used in membrane or separations applications.

1. A method for removing uremic toxins from blood, the method comprising:
exposing blood to iron-based metal-organic frameworks; and
allowing the metal-organic frameworks to bind a least one uremic toxin in the blood.

2. The method of claim 1, wherein after allowing the metal-organic frameworks to bind the least one uremic toxin, at least 70 wt % of p-cresyl sulfate is present in a bound state.

3. The method of claim 1, wherein the metal-organic frameworks are iron-based metal-organic frameworks.

4. The method of claim 1, wherein the metal-organic frameworks include Iron 1,3,5-benzenetricarboxylate (MIL-100(Fe)).

5. The method of claim 1, wherein the metal-organic frameworks are Iron 1,3,5-benzenetricarboxylate (MIL-100(Fe)).

6. The method of claim 1, wherein the metal-organic frameworks have benzenetricarboxylate (BTC) linkers.

7. The method of claim 1, wherein the blood is exposed to about 700 milligrams (mg) to about 800 mg of the metal-organic frameworks.

8. The method of claim 1, wherein after the metal-organic frameworks bind the at least one uremic toxin, the metal-organic frameworks are separated from the blood.

9. The method of claim 1, wherein the uremic toxin includes p-cresyl sulfate.

10. The method of claim 9, wherein the method is performed until the p-cresyl sulfate concentration of the blood is equal to or less than 10 μM.

11. A method for predicting the adsorptive capacity of a metal-organic framework for a uremic toxin, the method comprising:
determining uptake of the uremic toxin by the metal-organic framework as a function of mass of metal-organic frameworks while holding concentration of the uremic toxin in a solution and volume of the solution constant;
determining uptake of the uremic toxin by the metal-organic framework as a function of concentration of the uremic toxin in the solution while holding the mass of metal-organic frameworks and volume of the solution constant;
determining uptake of the uremic toxin by the metal-organic framework as a function of the solution volume while holding the mass of metal-organic frameworks and the concentration of the uremic toxin of the solution constant; and
performing multivariate linear regression to produce an uptake function as an effect of the solution volume, uremic toxin content and metal-organic framework mass.
